(19) United States
(12) Patent Application Publication
(10) Pub. No.: US 2014/0187834 A1
(43) Pub. Date: Jul. 3, 2014

(54) PROCESS FOR EXTRACTING C4+ OLEFINS AND A PROCESS FOR CONVERTING OXYGENATES TO OLEFINS

(71) Applicant: Shell Oil Company, Houston, TX (US)

(72) Inventors: Sivakumar SADASIVAN VIJAYAKUMARI, Amsterdam (NL); Jeroen VAN WESTRENEN, Amsterdam (NL)

(73) Assignee: Shell Oil Company, Houston, TX (US)

(21) Appl. No.: 14/136,021

(22) Filed: Dec. 20, 2013

(30) Foreign Application Priority Data
Dec. 31, 2012 (EP) 12199829.8

(51) Int. Cl. C07C 1/22; C07C 4/02

(52) U.S. Cl. CPC C07C 1/22; C07C 4/02
USPC 585/324; 585/639

(57) ABSTRACT

The current invention provides a process for extracting C4+ olefins from a stream comprising C4+ olefins and C4+ paraffins, wherein an oxygenate, preferably methanol (MeOH) is used as extractant, and wherein the resulting extract phase comprising C4+ olefins and extractant is converted into olefins. Also provided is a process for converting oxygenates to olefins, wherein the oxygenate preferably comprises MeOH, wherein the oxygenate is used as extractant and wherein an extract phase comprising C4+ olefins and the extractant are converted into olefins.

### This application claims the benefit of European Application No. 12199829.8 filed Dec. 31, 2012, which is incorporated herein by reference.

### FIELD OF THE INVENTION

The current invention provides a process for extracting C4+ olefins from a stream comprising C4+ olefins and C4+ paraffins, wherein methanol (MeOH) is used as extractant, and wherein the resulting extract phase comprising C4+ olefins and extractant is converted into olefins. Also provided is a process for converting oxygenates to olefins, wherein the oxygenate comprises MeOH, wherein the oxygenate is used as extractant and wherein an extract phase comprising C4+ olefins and the extractant are also converted into olefins.

Reference herein to a C4+ hydrocarbon is to hydrocarbons comprising 4 or more carbon atoms.

### BACKGROUND

Oxygenate-to-olefin (OTO) processes are used for producing light olefins: ethylene (C2=), propylene (C3=) and mixtures thereof. These light olefins are essential building blocks for the modern petrochemical and chemical industries. The search for alternative materials for light olefin production has led to the use of oxygenates such as alcohols and, more particularly, to the use of methanol (MeOH), and or ethanol, or their derivatives such as dimethyl ether (DME), for example. Common oxygenate feedstocks are methanol (MeOH) and/or dimethylether (DME), in which case the process is referred to as a methanol-to-olefin process. In the OTO process, the oxygenate is converted in an oxygenate-to-olefin reactor to ethylene and propylene using a suitable conversion catalyst. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

The conversion of alcohols is for instance described in U.S. Pat. No. 3,894,107. It is generally known that the process can be optimized to produce a major fraction of C2-C3 olefins. However, also paraffins, aromatics, naphthenes and higher olefins can be formed by hydrogen transfer, alkylation and oligomerisation. Prior process proposals have included a separation section to recover ethylene and propylene from the reaction effluent.

As indicated, such processing typically produces or results in a range of olefin reaction products as well as unreacted oxygenates, oxygenate derivatives, and other trace oxygenates. Typical or common OTO processing schemes include an oxygenate absorber whereby water (preferably at a temperature in the range of 25 to 40° C.) is used to absorb oxygenates, e.g., methanol and DME, from the light olefin product, while cooling down the product reactor effluent. This oxygenate-containing circulated water is subsequently stripped in an oxygenate stripper to recover methanol and DME, with such recovered materials ultimately recycled to the oxygenate conversion reactor. The oxygenate conversion product stream resulting from the oxygenate absorber is typically passed to one or more compressors. Moreover, it is treated, for instance between the 4^{th} and 5^{th} stage of compression, in a CO2 removal zone wherein the dewatered oxygenate conversion product stream is contacted with caustic solution to remove carbon dioxide.

From for instance WO 2008/039552 and U.S. Pat. No. 5,914,433 it is known that the amounts of light olefins resulting from such oxygenate to olefin processing can be further increased by reacting, i.e., cracking, heavier hydrocarbon products, particularly heavier olefins such as C4 and C5 olefins, hereinafter C4+ olefins to light olefins. The latter reference discloses a process wherein a propylene stream and/or mixed butylene is fractionated from said light olefin stream and cracked to enhance the yield of ethylene and propylene products.

Whereas in the processes above the cracking is carried out in a separate reaction zone, it is also possible to combine the OTO process with the cracking of a recycle stream in the same reaction zone. Such a process is for instance described in U.S. Pat. No. 6,441,261.

Either way, the integration of an olefin cracking processing advantageously monetizes the C4+ olefins via conversion of a substantial portion of such C4+ olefins to generally more commercially desirable propylene and ethylene. This of significant economic interest. However, in order to use the stream containing C4+ olefins, C4 paraffins and the like should be removed, at least in part. Their presence, for instance in a recycle stream adversely affects the economics of the process. Moreover, being largely inert, such paraffins would build up in a recycle stream.

In view thereof, there is an on-going need and a demand for a process wherein olefins, in particular C4+ olefins are effectively extracted from a stream comprising the same. Moreover, there is a need to do so such that the extract phase can be used effectively and with little capital expenditure and/or operating costs, to generate light olefins in an economic manner.

From GB 881597 a process for separating butylenes from butane is known. In this process, the C4 olefins and butane in a mixture are separated by a combination of solvent extraction and fractional distillation. Suitable solvents include various mono- and polyhydric alcohols (e.g., methanol); ethers; nitriles; esters; ketones, aldehydes and other organic solvents. Aqueous methanol solutions containing from 2 to 30% by weight water are amongst the particularly noteworthy examples of useful solvents in that process. The process comprises contacting the mixture in an extraction zone with a solvent which has a preferential solubility for butenes relative to butane and which boils at a temperature substantially above the boiling point of the mixture, separately withdrawing from the extraction zone a raffinate product containing a high concentration of butane and an extract phase comprising solvent containing dissolved butenes and butane, and subjecting said extract phase to a stripping operation. The extract phase is then divided into an overhead fraction containing butene-1 and butane, a side-cut product fraction containing a high concentration of butene-2 and a residual solvent fraction substantially free from hydrocarbons. The separation of the solvent and C4 olefins is therefore an essential feature of this process. There is nothing in this document suggesting the use of the extract phase as such.

### Summary of Invention

The current invention provides a process for extracting C4+ olefins from a stream comprising C4+ olefins and C4+ paraffins, wherein an oxygenate, preferably a C1-C4 alcohol, more preferably methanol (MeOH) is used as extractant, and wherein the resulting extract phase comprising C4+ olefins and extractant is converted into olefins. The invention also provides a process for converting oxygenates to olefins, wherein the oxygenate comprises a C1-C4 alcohol, more preferably MeOH, wherein the oxygenate is used as extractant and wherein an extract phase comprising C4+ olefins and the extractant are also converted into olefins

### BRIEF DESCRIPTION OF DRAWINGS

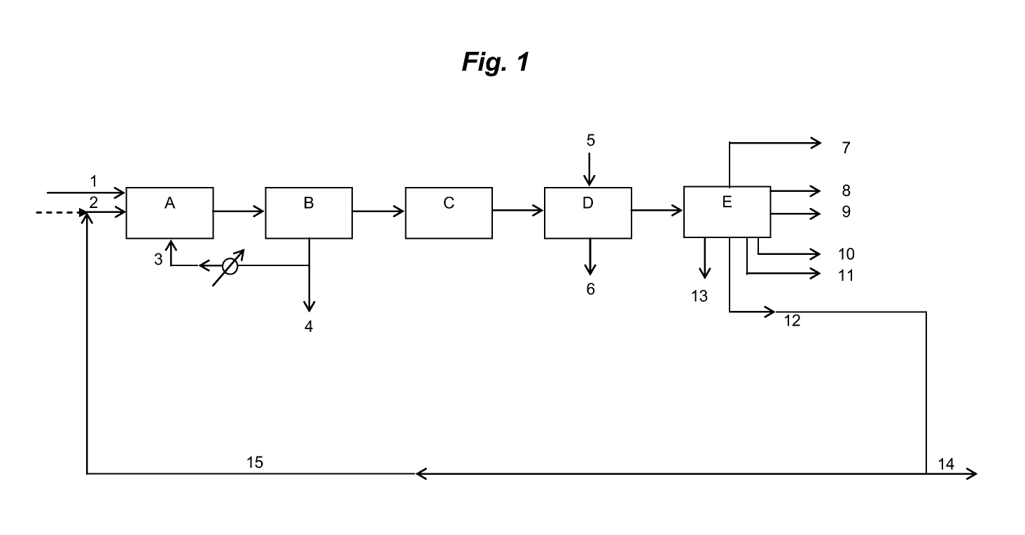

The attached FIG. 1 shows an example of a flow scheme of a conventional process with a C4 recycle stream (effluent of an OTO process, wherein at least the light olefins, C2= and C3=, have been removed) and having a bleed to avoid the build-up of C4 paraffins in the process.

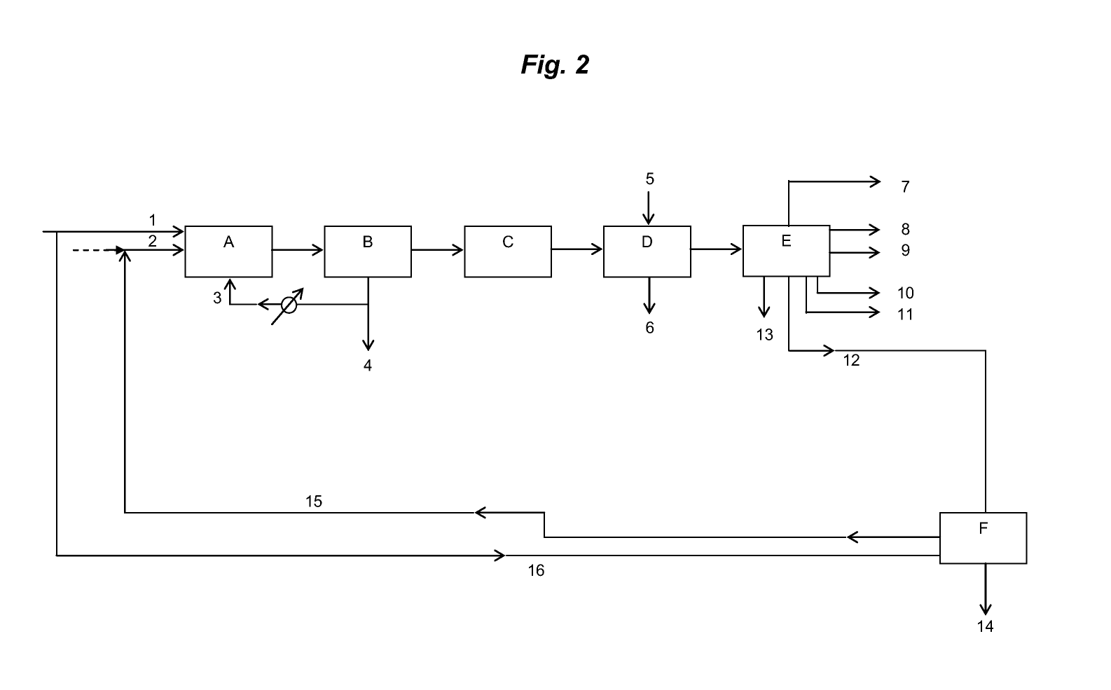

The attached FIG. 2 shows an example of a flow scheme of the invention in which a recycle stream (effluent of an OTO process, wherein at least the light olefins, C2= and C3=, have been removed) is treated with an extractant and the extract phase is fed to an OTO process.

In both Figures, unit A represents the OTO reactor. Oxygenate (e.g., MeOH) 1 and a stream 2 comprising C4+ olefins is introduced in the OTO reactor. The effluent is then treated in unit B, the quench tower. Process water 4 may be removed, whereas some steam 3 may be recycled. Next, the effluent is compressed in unit C. Subsequently, it is treated with caustic 5 in unit D to remove carbon dioxide 6. The product is then sent to a unit or series of units E, to split the product in a lights end 7, ethylene 8, ethane 9, propylene 10, and propane 11. In addition, there is a stream comprising C4 paraffins and C4 olefins 12, and a stream comprising “heavies” 13. In the conventional process of FIG. 1, the stream 12 is recycled, with a significant part being removed and lost as bleed stream 14. FIG. 2 is similar to FIG. 1, however now with a unit F, for extracting C4+ olefins. It uses a part stream 16 of the oxygenate stream 1. The olefin enriched oxygenate stream 15 is then recycled to the OTO unit A.

### DETAILED DESCRIPTION

Oxygenate to olefin, or OTO processes are well known in the art and are also referred to as methanol-to-olefins or methanol-to-propylene processes. In an OTO process, typically the oxygenate is contacted with a zeolite-comprising catalyst at elevated temperatures. In contact with the zeolite-comprising catalyst, the oxygenate is converted into ethylene and/or propylene. Besides ethylene and propylene, substantial amounts of C4+ olefins are produced as well as C4+ paraffins. To increase the total yield of ethylene and propylene, these C4+ olefins may be converted to obtain further ethylene and propylene. One way of converting the C4+ olefins to ethylene and propylene is through cracking the C4+ olefins by contacting the C4+ olefins at elevated temperature with a zeolite-comprising catalyst. This process is generally referred to as an olefin cracking process or OCP.

In the process according to the present invention, rather than sending the entire stream comprising the C4+ olefins obtained for instance from an OTO process to an olefin cracking process, it is preferred that the stream or a part thereof is treated by extraction.

In the process according to the present invention, the extraction is carried out with an oxygenate used as feedstock in OTO processes. Suitable oxygenates include methanol, ethanol, and in particular bio-ethanol, propanol and butanol, preferably with MeOH. The MeOH may contain other oxygenates, including (minor) amounts of higher alcohols. The use of an oxygenate has the advantage that the extract phase can be used without having to remove foreign components. Indeed, if the extract phase is used in an OTO process, then the extract phase can be used as feedstock with very little investment.

The MeOH can be used as such or as a mixture with other oxygenates. Preferably the extractant is MeOH, containing less than 30% wt on the total of other oxygenates, more preferably being the only oxygenate used in the process of the current invention. The extractant may comprise water, e.g., up to 30% wt of the extractant.

The C4+ containing stream may be the first effluent stream of an OTO process. This stream comprises at least ethylene and/or propylene.

Preferably the first effluent stream is first fractionated. It is common to send the first effluent stream first to a quench tower. Here typically most of the water and oxygenates like MeOH may be removed. Subsequently it is common to submit the first effluent stream to one or more compressors or compression stages; to an oxygenate stripper, and/or to a CO2 removal zone. Finally, it is common to send the first effluent stream to a drier. In the process of the current invention, these steps are preferably included, more preferably in the described order. For instance, an oxygenate stripper and/or CO2 removal zone may be part of a “compression train”. In this case, after CO2 removal the product gas is further compressed. Next it is common to fractionate the first effluent stream into a first product fraction comprising C2 and C3 hydrocarbons and a second fraction comprising the C4+ olefins. Also a series of distillation columns may be used. For instance, the first effluent stream, or the first fraction if a split has already been made, may be sent to a de-ethanizer, typically operating at 22-26 bars, wherein C2 hydrocarbons are isolated from the top. The C1-component therein may be compressed and further purified, if needed. The bottom stream after the de-ethanizer may then be sent to a depropanizer, typically operating at 12-21 bars, wherein C3 hydrocarbons are isolated from the top. The overhead of the de-ethanizer and of the depropanizer may be sent to a C2, where the ethylene and the ethane are split, respectively to a C3-splitter, where the propylene and the propane are split. The paraffins ethane and propane may be sent to a cracking furnace or sold to the LPG market. In addition to this configuration other process line-ups, e.g., using a de-propanizer or de-methanizer first, may be used without departing from the gist of the current invention.

Also preferably removed and isolated from the light end are the residual oxygenates. This fraction, comprising MeOH and/or DME, may be recycled. For instance, this fraction may be used together with fresh MeOH as extractant.

The stream containing C4+ olefins is preferably a stream essentially composed of C4+ hydrocarbons. In other words, it is preferably a stream wherein less than 5% wt, more preferably less than 1% wt of light ends remain.

It is also common to remove “heavies” from the first effluent stream. Heavies include aromatic compounds such as benzene, toluene and xylenes, that may be valuable in their own right. Although not described herein, these components are preferably removed from the stream containing the C4+ olefins as well. Indeed, part or all of the xylenes in the heavy fraction comprising aromatics may be withdrawn from the process as a product.

As mentioned herein above, at least part of the second fraction is reused, more preferably recycled back to the first reaction zone. The C4 olefins in the second fraction may react in the first reaction zone with methanol to generate additional ethylene and propylene. Alternatively, the C4 olefins may also be converted into ethylene and propylene at conditions more suitable for olefin cracking reactions.

As indicated above, the effluent of an OTO zone also comprises paraffinic hydrocarbons including C4 paraffins. The C4 paraffins cannot be separated through conventional distillation. When these C4 paraffins are recycled to the first reaction zone as part of the second fraction, or to a unit operating at olefin cracking conditions, this may lead to a build-up of a paraffin content in the second fractions, as the C4 paraffins do not, or not to any appreciable extent, react in the first reaction zone under OTO conditions and thus pass through the first reaction zone unconverted. In the process of the current invention, therefore at least part of the paraffins in the second fraction is purged from the process. This may be done by withdrawing a certain part of the second fraction, such as between 1 and 15 wt% based on the second fraction, as a purge or a bleed stream.

Another alternative may include the use of extractive distillation with polar liquids to separate the olefins and paraffins in the second fraction. In this way, a paraffin-enriched stream may be purged, while an extract phase enriched in olefin is recycled.

In the process of the current invention, the separation of the olefins and paraffins in the second fraction or in the bleed stream is carried out with oxygenates, more preferably with MeOH, or a mixture thereof, as extractant. It is observed here that using a purge or bleed stream is the conventional manner to avoid build-up of paraffins. However, this stream will still comprise valuable C4+ olefins. The process of the current invention can therefore be economically used both for the second fraction as well as for the bleed stream.

Preferably, the extracted C4+ olefins are provided to the first reaction zone as part of the oxygenate feed, preferably in a process that combines the oxygenate-to-olefin process and the olefin cracking process in one reaction zone. Also within the scope of the invention is the use of a combination of the OTO process with a zone operated under olefin cracking conditions. Additional C4+ olefins or oxygenates may be added to the feedstock to optimize the same for this reaction.

Suitable conditions to extract C4+ olefins from a stream further comprising paraffins are known. In the GB 881597 cited above, incorporated herewith by reference, extraction is carried out in a liquid-liquid extraction column. This column is preferably designed for counter-current extraction, with the feed being introduced below the point at which the extractant is charged therein.

The stream comprising C4+ olefins and C4+ paraffins (C4 feed) is preferably liquefied. This step too is conventional.

The extraction column is preferably designed to provide maximum contact between the C4 feed and the extractant. Typical process conditions utilizable in this liquid-liquid extraction step include a solvent to hydrocarbon volumetric feed ratio of from about 0.2 to 1 to about 40 to 1, preferably of more than 1 volume of solvent to 1 volume of the C4 feed.

As is known from GB 881597, when utilizing an aqueous methanol solution containing approximately 10% by weight of water, an extractant to feed ratio of about 15 to 1 is especially suitable in conjunction with the operating temperature of about 30° C. and a pressure of about 20 atmospheres.

Preferably, however, extractive distillation is used. In extractive distillation preferably a much smaller solvent (=extractant) to hydrocarbon (=stream comprising C4+ olefins and C4+ paraffins) volumetric feed ratio is used. In the preferred case of extractive distillation, the solvent to hydrocarbon volumetric feed ratio is preferably from 0.02 to 1 to 0.5 to 1, more preferably from 0.05 to 1 to 0.2 to 1, still more preferably about 0.1 to 1. The extractive distillation column may be of any suitable design-sufficient to obtain efficient vapor-liquid contact. It could be a packed or trayed column. It may be packed with typical contacting materials, such as quartz chips, Berl saddles, ceramic helices and other packaging materials well-known in the art. Typically, the extract phase is recovered from the bottom of the distillation column, with the C4+ paraffinic fraction being recovered from the top of the column as distillate.

If the extract phase is used in an OTO process that is combined with the olefin cracking process in one reaction zone, then the oxygenate feed preferably comprises at least 50 wt %, of C4 olefins, more preferably at least 75 wt %, even more preferably at least 90 wt % of C4 olefins, based on the total amount of olefins in the oxygenate feed.

The OTO and OCP processes are described in more detail herein below.

The oxygenate-to-olefin process is a process for preparing ethylene and/or propylene. In this process an oxygenate feed is provided to a first reaction zone. The oxygenate feed comprises MeOH as oxygenate. Preferably, the feed further comprises C4+ olefins. The conditions described hereafter are the conditions of a typical OTO process. Reference herein to the oxygenate feed is to a single feed comprising oxygenate and C4+ olefins or to two or more sub feed each comprising one or more of the compounds of the oxygenate feed, which combined form the oxygenate feed. For instance the oxygenate feed may be provided as sub feed comprising oxygenate and a sub feed comprising C4+ olefins.

Preferably, the oxygenate feed in an OTO process that includes an olefin cracking process in the same reaction zone comprises oxygenate and olefins in an oxygenate:olefin molar ratio in the range of from 1000:1 to 1:1, preferably 100:1 to 1:1. More preferably, in a oxygenate:olefin molar ratio in the range of from 20:1 to 1:1, more preferably in the range of 18:1 to 1:1, still more preferably in the range of 15:1 to 1:1, even still more preferably in the range of 12:1 to 1:1. It is preferred to convert a C4+ olefin together with an oxygenate, to obtain a high yield of ethylene and propylene. For instance, preferably at least one mole of MeOH is provided for every mole of C4+ olefin. DME may be prepared in the reaction zone from 2 moles of MeOH. Thus, in case of DME, at least 0.5 mole of DME is provided for every mole of C4+ olefin.

In the first reaction zone, the oxygenate-comprising feed is contacted with a suitable catalyst. This may, for instance, be a zeolite-comprising catalyst. Catalysts suitable for converting the oxygenate feedstock comprise one or more molecular sieves. Such molecular sieve-comprising catalysts typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing tetrahedral units, more preferably, two or more [SiO4], [AlO4] and/or [PO4] tetrahedral units. These silicon, aluminum and/or phosphorus based molecular sieves and metal containing silicon, aluminum and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable molecular sieves are silicoaluminophosphates (SAPO), such as SAPO-17, -18, 34, -35, -44, but also SAPO-5, -8, -11, -20, -31, -36, 37, -40, -41, -42, -47 and -56; aluminophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIB, VIIB, VIIIB and Lanthanides of the Periodic Table of Elements. Preferably Me is selected from one of the group consisting of Co, Cr, Cu, Fe, Ga, Ge, Mg, Mn, Ni, Sn, Ti, Zn and Zr.

Alternatively, the conversion of the oxygenate feedstock may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, and the FER type. Other suitable zeolites are for example zeolites of the STF-type, such as SSZ-35, the SFF type, such as SSZ-44 and the EU-2 type, such as ZSM-48.

Aluminosilicate-comprising catalyst, and in particular zeolite-comprising catalyst are preferred when an olefinic co-feed is fed to the oxygenate conversion zone together with oxygenate, for increased production of ethylene and propylene.

Preferred catalysts for OTO processes comprise SAPO, MEL and/or MFI type molecular sieves, whereby the latter two are zeolite molecular sieves. More preferred catalyst comprise SAPO-34, ZSM-11 and/or ZSM-5 type molecular sieves. A preferred MFI-type zeolite for the OTO catalyst has a silica-to-alumina ratio, SAR, of at least 60, preferably at least 80. More preferred MFI-type zeolite has a silica-to-alumina ratio, SAR, in the range of 60 to 150, preferably in the range of 80 to 100.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus.

The oxygenate-comprising feed is contacted with the catalyst at a temperature in the range of from 350 to 1000° C., preferably of from 450 to 650° C., more preferably of from 530 to 620° C., even more preferably of from 580 to 610° C.; and a pressure in the range of from 0.1 kPa (1 mbar) to 5 MPa (50 bar), preferably of from 100 kPa (1 bar) to 1.5 MPa (15 bar), more preferably of from 100 kPa (1 bar) to 300 kPa (3 bar). Reference herein to pressures is to absolute pressures.

Obviously, it is also possible to use several reactors in series, wherein the effluent of a first reactor is used as feed in a subsequent reactor. It is therefore within the scope of the present invention that the extract phase is used, not in an OTO process with a reaction zone where olefin cracking occurs, but in a combination of an OTO process with a separate process for cracking the C4+ olefins. This may be done in an OCP reactor using appropriate catalysts, similar to the catalysts mentioned above, and appropriate olefin cracking reaction conditions. For instance, from US6303839, the contents of which are hereby incorporated, it is known to fractionate a propylene stream and/or mixed butylene from a light olefin stream and to crack the same to enhance the yield of ethylene and propylene products. This combination of light olefin product and propylene and butylene cracking in a riser cracking zone or a separate cracking zone provides flexibility to the process.

In this case, the extract phase used as feed is contacted with the catalyst at a temperature in the range of from 350 to 1000° C., preferably of from 450 to 650° C., more preferably of from 530 to 620° C., even more preferably of from 580 to 610° C.; and a pressure in the range of from 0.1 kPa (1 mbar) to 5 MPa (50 bar), preferably of from 100 kPa (1 bar) to 1.5 MPa (15 bar), more preferably of from 100 kPa (1 bar) to 300 kPa (3 bar). Reference herein to pressures is to absolute pressures.

### EXAMPLE

In a conventional set-up wherein an oxygenate is converted into an olefin, anywhere between 5 and 30% by volume of the fraction containing C4+ paraffins and C4+ olefins is removed as a bleed. As a result, between 5 and 30% of the valuable C4+ olefins are removed as well. This loss can easily be reduced by 50%.

In an experiment simulated using Aspen Plus® a bleed stream comprising 74% by volume of C4 saturates and 26% by volume of C4 olefins is extracted with MeOH in an Extractive Distillation (ED) column. The ED column has 60 stages and was operated at a column pressure of 6 bara, and a reflux ratio of 3. A solvent to feed volumetric ratio of 0.1 is used. By using the process of the invention, in this case with extractive distillation of the bleed at a solvent to feed volumetric ratio of 0.1, the loss of C4 olefins is shown to be reduced by 50%.

### INDUSTRIAL APPLICABILITY

The process of the current invention is particularly useful in the preparation of the light olefins, ethylene and propylene.

What is claimed is:

1. A process for extracting C4+ olefins from a stream comprising C4+ olefins and C4+ paraffins, wherein an oxygenate or mixture of oxygenates is used as extractant, and wherein the resulting extract phase comprising C4+ olefins and extractant is converted into olefins.

2. The process of claim 1, wherein the oxygenate is a C1-C4 alcohol or mixture thereof.

3. The process of claim 1, wherein the stream comprising C4+ olefins and C4+ paraffins is an effluent stream or a recycle stream or part of a bleed stream in an Oxygenate-to-Olefin (OTO) process.

4. The process of claim 3, wherein the effluent stream is first treated in a quench tower to remove water and/or any remaining oxygenates.

5. The process of claim 4, wherein the removed oxygenates are recycled.

6. The process of claim 3, wherein an aromatic containing stream is removed from the effluent stream.

7. The process of claim 1, wherein an extractive distillation is used.

8. The process of claim 7, wherein the volumetric feed ratio of the extractant to the stream comprising C4+ olefins and C4+ paraffins is from 0.02 to 1 to 0.5 to 1.

9. A process for converting oxygenates to olefins, wherein the oxygenate comprises a C1-C4 alcohol or mixture thereof, preferably MeOH, wherein the oxygenate is used as extractant and wherein an extract phase comprising C4+ olefins and the extractant are converted into olefins.

10. The process of claim 9, wherein the extract phase is recycled to a reaction zone combining an OTO process with a zone operated under olefin cracking process (OCP) conditions.
